(19) United States
(12) Patent Application Publication
(10) Pub. No.: US 2023/0220353 A1
(43) Pub. Date: Jul. 13, 2023

(54) IN VITRO LIVER ORGANOIDS AND MINI-BILE DUCT MODELS OF BILIARY ARTRESIA AND APPLICATIONS THEREOF

(71) Applicants: Hai Bing YUE, Hong Kong (CN); Chi Hang LUI, Hong Kong (CN); Kwong Hang TAM, Hong Kong (CN)

(72) Inventors: Hai Bing YUE, Hong Kong (CN); Chi Hang LUI, Hong Kong (CN); Kwong Hang TAM, Hong Kong (CN)

(21) Appl. No.: 17/573,371

(22) Filed: Jan. 11, 2022

(51) Int. Cl. C12N 5/071

(52) U.S. Cl. CPC C12N 5/0671; C12N 2513/00; C12N 2506/45; C12N 2501/998; C12N 2501/999

(57) ABSTRACT

The present disclosure relates to in vitro models of biliary atresia obtained by culturing of human liver organoids and/or mini-bile ducts and exposing the liver organoids and/or mini-bile ducts to biliatresone. The present disclosure also provides methods of preparation of the in vitro models of biliary atresia, and applications thereof.

### FIELD OF THE DISCLOSURE

The present disclosure relates to in vitro models of biliary atresia, methods of preparation and applications thereof.

### BACKGROUND OF THE DISCLOSURE

Biliary atresia (BA) is a rare and poorly understood and devastating obstructive bile duct disease of newborns. It is an inflammatory cholangiopathy characterized by biliary cirrhosis, fibrotic disorder, neonatal cholestasis and inflammatory obstruction of the biliary tract (1, 2). Kasai surgery is the first-line treatment for BA patients, which replaces the obliterated extrahepatic bile duct with an intestinal conduit to re-establish bile flow (for review see (3)).

For many BA patients, liver transplantation however is the final outcome (4). BA affects 5-20:100,000 live births. Over 90% of the cases are non-familial and without a clear genetic cause (for review see (5)). Susceptibility loci have been identified on chromosomes 10q24.2 (6) and 2q37.3 (7), and later, the ADD3 and EFEMP1 genes were identified as aberrantly regulated in BA (8, 9). Polycystic kidney disease 1 like 1 Gene (PKD1 L1) gene mutations were also identified in patients with BA splenic malformation syndrome (10). It is likely that BA has multiple etiologies, and viral infections (11), exposure to toxins (12) and a dysfunctional immune response (13) are likely to contribute by causing inflammation and driving BA pathogenesis.

Biliatresone is a naturally occurring isoflavonoid-related 1,2-diaryl-2-propenone plant toxin found in Dysphania glomulifera and D. littoralis. This plant toxin has been implicated in BA-like syndrome outbreaks in Australian livestock also causes bile duct destruction in zebrafish (12), mouse cholangiocyte spheroids and ex-vivo bile duct culture (14), and in mouse neonates (15). However, there has been no direct evidence for the hypothesis that biliatresone exposure has an etiological/pathobiological role for BA in humans, therefore the relevance of biliatresone in humans remains unknown.

There is a need to gain better understanding of BA and other bile duct diseases, the effects of other toxins on liver, as well as a need to develop useful therapeutic treatments for BA and other bile duct diseases. Currently, cell lines and animal models are commonly used in toxicity testing. However, as cell lines and model animals do not resemble normal cell physiology and metabolism of humans; screening using these cells/model animals have limited relevance for the toxicities in humans.

A liver organ contains mainly liver cells (hepatocytes) and bile duct cells (cholangiocytes). Despite accounting for 5% of cells in the liver, cholangiocytes (bile duct cells) are essential for health; biliary tract disorders account for 70% and 33% liver transplantations in children and adults respectively.

Bile ducts are the system that collects bile synthesized from the liver cells, and transports the bile from the liver to the gut. Defective function of the bile duct/bile duct cells lead to bile duct diseases. In addition, bile duct cells modify the bile content, and are implicated in the bile duct diseases. Previous studies primarily focus on liver cells, and research on the bile duct are limited and therefore, the physiological functions of bile duct cells in healthy and disease condition is an area that has not been well studied and described.

Cells from the cholangiocyte lineage of mice and humans can be cultured in the form of 3 dimensional organoids (16-19). Liver organoids are composed of cholangiocytes and hepatocytes, but with cholangiocytes as a major cell type in the organoids.

Using an organoid approach, liver organoids from BA liver and RRA-BA (Rhesus rotavirus A-infected mouse) mouse liver were previously generated. It was reported that (a) liver organoids from BA patients and a BA mouse model exhibited aberrant morphology and disturbed apical-basal organization; (b) a defective cholangiocyte development and altered beta-amyloid-related gene expression is seen in BA organoids; (c) β-amyloid peptide (Aβ) accumulation is observed in BA livers and exposure to Aβ induced the aberrant morphology in control organoids (18). The aberrant organoid morphology observed in liver organoids derived from BA liver is believed to be specific for BA.

The previously reported liver organoids derived from BA patients with BA anomalies are valuable for the examination of BA and the drug development purposes, however, practical challenges are involved in obtaining by surgery a sufficient amount of liver tissues from newborns with BA.

As compared to cell lines and animal models that have limited relevance for toxicities in humans, human derived cells are preferable in mimicking native physiology and modelling disease, and human derived cells are more useful for performing toxicity and drug screening. Therefore, there remains to a need for in vitro BA-specific liver organoids and mini-bile duct for use in bile duct toxicity screening and development of anti-toxicity treatments, and a method of supplying such in vitro BA-specific liver organoids and mini-bile duct in a sufficient amount in a reproducible manner.

It is an object of the present disclosure to address or at least partially ameliorate some of the above problems of the current approaches.

It is an object of the present disclosure to provide in vitro disease-specific liver organoids and mini-bile duct, including liver organoids and mini-bile duct with BA characteristics.

It is an object of the present disclosure to provide methods for preparing liver organoids and mini-bile duct model of BA.

It is an object of the present disclosure to provide a platform substantially useful for toxicity screening and/or development of anti-toxicity treatment.

### SUMMARY OF THE DISCLOSURE

Features and advantages of the disclosure will be set forth in the description which follows, and in part will be obvious from the description, or can be learned by practice of the herein disclosed principles. The features and advantages of the disclosure can be realized and obtained by means of the instruments and combinations particularly pointed out in the appended claims.

In accordance with a first aspect of the present disclosure, there is provided an in vitro liver organoid model of biliary artresia comprising biliatresone-treated liver organoids and/or mini-bile ducts.

In a one embodiment, the liver organoids and/or mini-bile ducts are characterized by one or more characteristics selected from the group consisting of retarded growth; disturbed apical-basal organization; defective cholangiocyte development; β-amyloid (Aβ) accumulation; reduction of primary cilia and cilia mechano-sensory function of cholangiocytes.

In another embodiment the biliatresone-treated liver organoids and/or mini-bile ducts are characterized by one or more characteristics relative to the untreated cells, wherein the characteristics are selected from the group consisting of:
reduced expression of cholangiocyte marker CK19;
increased expression of marker HFN4A;
reduced ZO-1 immunoreactivity;
ectopic expression of F-actin at both of the apical and basal sides;
reduced primary cilia; and
reduced cilia mechano-sensory function of cholangiocytes.

Preferably, the biliatresone-treated liver organoids and/or mini-bile ducts are derived from a non-diseased liver tissue or human induced pluripotent stem cells (IPSCs).

In one embodiment, the liver organoids and/or mini-bile ducts are obtained by the following steps:

a. obtaining liver tissues from non-diseased human subjects;

b. culturing CD326 positive cells in an extracellular protein matrix in the presence of a culture medium;

c. contacting the cells obtained from the culturing step with biliatresone.

In an alternative embodiment, the liver organoids and/or mini-bile ducts are obtained by:

a. generating human induced pluripotent stem cells (hIPSCs) from peripheral blood;

b. differentiating hIPSCs to liver organoids;

c. contacting the liver organoids obtained from the differentiating step with biliatresone.

Advantageously, the liver organoids and/or mini-bile ducts are contacted with biliatresone at a concentration of 1 μg/ml-10 μg/ml, preferably 2 μg/ml-5 μg/ml; and the liver organoids are contacted with bilitresone for 1 to 10 days, preferably 3 to 5 days.

In a preferred embodiment, the model of the present disclosure is a three-dimensional (3D) liver organoid model of biliary artresia, which may comprise only one or more organoid(s) derived from liver or hIPSCs.

Advantageously, the model of the present disclosure may comprise both liver organoids and mini-bile ducts derived from liver or hIPSCs. Optionally, the model of the present disclosure comprises additional cell types selected from liver cells, vascular cells and immune cells.

In accordance with a second aspect of the present disclosure, there is provided an method of generating in vitro liver organoid model of biliary artresia comprising biliatresone-treated liver organoids and/or mini-bile ducts, the method comprises the steps of:

a. obtaining human liver tissue from subjects free of liver diseases;
b. digesting the tissue and selecting CD326 positive cells;
c. culturing CD326 positive cells in an extracellular protein matrix in the presence of a culture medium; and
d. contacting the cells obtained from the culturing step with biliatresone.

Advantageously, wherein the liver organoids and/or bile duct organoids are contacted with biliatresone at a concentration of 1 μg/ml-10 μg/ml, preferably 2 μg/ml-5 μg/ml; and the liver organoids are contacted with bilitresone for 1 to 10 days, preferably 3 to 5 days.

In a preferred embodiment, the culture medium comprises DMEM/F12, HEPES, Penicillin/Streptomycin, Amphotericin B, N2, B27, N-Acetylcysteine, gastrin, and growth factors: mEGF, FGF10, HGF, Nicotinamide, A83.01, FSK, Noggin, R-Spondin 1, Wnt3a, and Y27632. In accordance with a third aspect of the present disclosure, the use of the in vitro liver organoid model of biliary artresia according to claim 1 as a platform for screening for a toxin is provided.

In accordance with a four aspect of the present disclosure, the use of the in vitro liver organoid model of biliary artresia according to claim 1 as a platform for screening for an anti-toxin therapeutic.

In accordance with a fifth aspect of the present disclosure, there is provided a method of determining the effect of a test compound comprising the following steps:

a. providing the in vitro liver organoid model of biliary artresia according to claim 1;
b. contacting the in vitro liver organoid model of biliary artresia with the test compound.
c. determining the effect of the test compound on the liver organoids and/or min-bile ducts.

Preferably, the step of determining the effect of the test compound in the above method comprises quantification of one or more of the following parameters: growth rate, apoptosis, organoid polarity, organoid transportation, marker expression and functional maturity of the cells.

### BRIEF DESCRIPTION OF THE DRAWINGS

In order to describe the manner in which the above-recited and other advantages and features of the disclosure can be obtained, a more particular description of the principles briefly described above will be rendered by reference to specific embodiments thereof which are illustrated in the appended Figures. Understanding that these Figures depict only exemplary embodiments of the disclosure and are not therefore to be considered to be limiting of its scope, the principles herein are described and explained with additional specificity and detail through the use of the accompanying Figures.

Preferred embodiments of the present disclosure will be explained in further detail below by way of examples and with reference to the accompanying Figures, in which:—

FIG. 1 depicts a schematic diagram of the generation of biliatresone treated human liver organoids.

FIG. 2 depicts day-5 biliatresone-treated organoids stained for hematoxylin and eosin as compared to control.

FIG. 3A depicts day-2 biliatresone-treated human liver organoids stained for HNF4A (green) as compared to control.

FIG. 3B depicts day-2 biliatresone-treated human liver organoids stained for MDR1 (red) as compared to control.

FIGS. 3C-3D depict the results of MDR1 activity assay of the organoids. Top figure of FIG. 3C depicts the localization of fluorescent substrate Rhodamine 123 (R123) in day-2 biliatresone-treated organoids and the observed fluorescent intensity at 0 and 30 minutes of incubation. The results of control are shown in the lower figure of FIG. 3C. Upper figure of Top portion of FIG. 3D depict the localization of fluorescent substrate Rhodamine 123 (R123) in day-2 biliatresone-treated organoids and the observed fluorescent intensity at 0 and 30 minutes of incubation in the presence and absence of Verapamil. The corresponding results for a control are shown in FIG. 3D.

FIG. 4 depicts day-2 biliatresone-treated human liver organoids stained for ZO-1 and CK19 (bottom row) as compared to control (top row).

FIG. 5A depicts the localization of FITC-dextran in day-2 biliatresone-treated organoids at 10 and 30 minutes of incubation as compared to control.

FIG. 5B depicts the fluorescent intensity along the white line of FIG. 5A.

FIG. 6A depicts day-2 biliatresone-treated human liver organoids stained for protein F-actin (green) and β-amyloid (red) as compared to control. Boxed regions were magnified and shown as insets.

FIG. 6B is a bar chart showing the percentages of β-amyloid positive cells in day-2 biliatresone-treated organoids as compared to control.

FIG. 7A depicts day-1, -2-3 and -5 biliatresone-treated human liver organoids stained for pericentrin (PCNT; green) and acetylated α-tubulin (Tubulin; red) as compared to control.

FIG. 7B is a bar chart showing the percentages of ciliated cells in biliatresone-treated organoids as compared to control.

FIG. 8A depicts the perfusion setup for the microfluidic chip with human cholangiocyte monolayer.

FIG. 8B depicts the cholangiocyte monolayer cultured in the channel of microfluidic chip immunostained for ZO-1 (green).

FIG. 8C depicts cholangiocytes stained with Calbryte 520 before and after perfusion stimulation.

FIG. 8D is a bar chart showing the fluorescent intensity changes of highlighted region (ROI: region of interest) for 20 seconds after perfusion stimulation.

FIG. 9A depicts biliatresone-treated human cholangiocytes stained with Calbryte 520 as compared to control.

FIG. 9B depicts the profile of fluorescent intensity changes of selected control and biliatresone-treated human cholangiocytes (highlighted with dotted line) for 20 seconds.

FIG. 9C is a bar chart showing the percentage of evoked cells after perfusion stimulation of control (Ctrl) and biliatresone-treated (BTS) human cholangiocytes.

FIG. 10A depicts relative expression of SOX17 to GAPDH of 2 days and 5 days biliatresone-treated and untreated (Ctrl) human cholangiocytes.

FIG. 10B depicts GSH concentrations of biliatresone-treated and untreated (Ctrl) human cholangiocytes at 0, 1, 3, 6, 24 and 48 hours of treatment.

### DETAILED DESCRIPTION OF THE PREFERRED EMBODIMENTS

Various embodiments of the disclosure are discussed in detail below. While specific implementations are discussed, it should be understood that this is done for illustration purposes only. A person skilled in the relevant art will recognize that other components and configurations may be used without departing from the spirit and scope of the disclosure.

The etiology and pathogenesis of BA remain unknown, and the diagnosis and management of BA have not advanced significantly in the past years. Due to the severity of the BA in infants and the poor prognosis of the disease without surgical intervention, there is a need to better understand the disease to allow early diagnosis and development of new treatment options.

Biliatresone, a plant isoflavonoid-related 1,2-diaryl-2-propenone has been implicated in BA-like syndrome outbreaks in animals and yet the relevance of biliatresone in humans remains unknown. It is unexpectedly discovered and described herein that, upon a specific biliatresone toxin treatment, it is possible to generate human liver organoids and organoid-derived mini-bile ducts that exhibit BA-like anomalies. In particular, as illustrated in the Examples, biliatresone induced dysmorphogenesis; disturbed apical-basal organization; defective cholangiocyte development; β-amyloid (Aβ) accumulation; reduction of primary cilia and cilia mechano-sensory function of cholangiocytes.

These human liver tissue-derived BA-specific cells are able to mimic native physiology and are highly valuable in modelling the disease, and further applications. Accordingly, the present disclosure is able to provide “human bile duct in a dish”, a platform useful for toxicity screening and anti-toxicity treatment. The disclosed in vitro models are expected to improve the reliability of the toxicity screening and the efficacy for the development of anti-toxicity treatments. The liver organoid and its derived bile ducts provide a human proxy for toxicity screening of natural toxins, chemicals, drugs and herbal medicines that induce bile duct damages relevant for BA and other bile duct diseases.

As described herein, “in vitro BA model”, “in vitro model of BA” are used interchangeably and refer to cells, e.g. liver organoids, bile duct organoids, and mini-bile duct that have been treated with biliatresone. These cells may be derived from progenitor cells of liver biopsy of non-disease human or established using human induced pluripotent stem cells (IPSCs) via in vitro differentiation. Methods of generating human induced pluripotent stem cells (hIPSCs) from peripheral blood, and differentiating hIPSCs to liver organoids are known to those skilled in the art and described in Sampaziotis, F., M. C. de Brito, I. Geti, A. Bertero, N. R. Hannan and L. Vallier (2017). “Directed differentiation of human induced pluripotent stem cells into functional cholangiocyte-like cells.” Nat Protoc 12(4): 814-827.

In particular, the in vitro cells exhibit aberrant organoid morphology and other characteristics observed in liver organoids derived from BA liver. These cells may also be referred herein as “BA-specific cells”, “BA-specific liver organoids”, “BA-specific bile duct organoids”, “BA-specific mini-bile duct” and are described as displaying BA anomalies.

By using specific cell culture conditions, tissues/IPSCs can be steered to form two types of organoid that is either rich in hepatocytes or rich in bile duct cells (i.e. cholangiocytes). “Liver organoids” as described herein could refer to one of these two major types of organoid derived from tissues/IPSCs.

“Bile duct organoids” as described herein refer to organoids mainly comprising of bile duct cells (i.e. cholangiocytes). Bile duct organoids are spheroids with the apical side facing inside and the basal side facing outside. This may pose challenges for assessing the effect of toxins on the apical side of the bile duct cell layer.

The term “mini-bile duct” as described herein is used to represent a structure formed by bile duct cells with an in vivo arrangement (i.e. a one layer of bile duct cells with the apical side of the bile duct cells facing at one side, whereas the basal side of all the bile duct cells facing the opposite side. The apical side is the bile duct lumen facing side in vivo (the side bathing in the bile), therefore, the mini-bile duct would allow an easy manipulation of the “luminal” content and to address the effects of agents/different bile content on the behavior/physiological functions of the bile duct. This correct apical basal setup is crucial for bile duct functions. With such apical basal aspect of the mini-bile duct, “the close to in vivo” behavior and physiological functions of bile duct can be addressed readily. The effects of toxins on the bile duct epithelium can also been directly assessed.

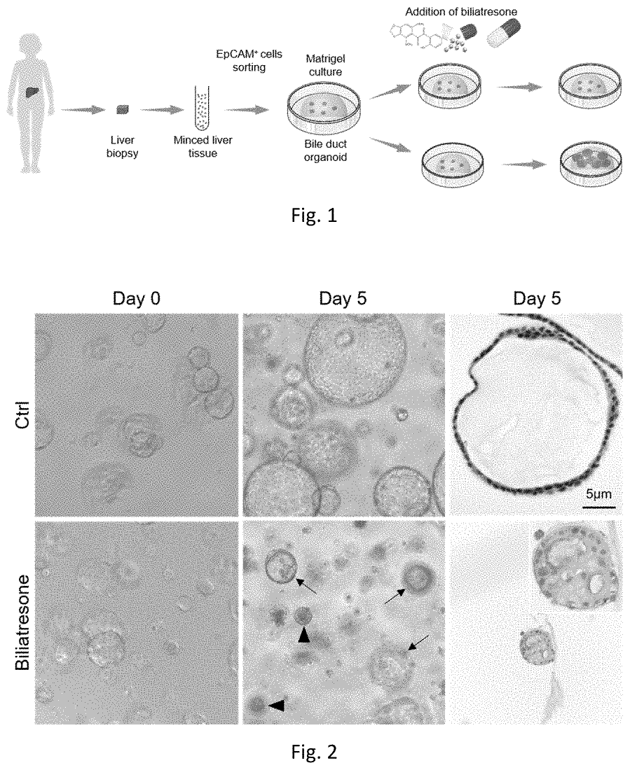

Referring to FIG. 1, there is shown a schematic diagram of the preparation of human liver organoids for biliatresone treatment. Liver biopsies of children with non-BA liver diseases including non-tumor liver of hepatoblastoma and choledochal cyst were obtained for liver organoids generation using a modified protocol based on a previously reported method (18).

Briefly, liver biopsy was minced and digested, hepatoblasts/cholangiocyte progenitors were enriched by cell sorting by using surface markers such as EpCAM, and sorted cells were filtered. Hepatoblasts/cholangiocyte progenitors were mixed with an extracellular protein matrix e.g., Matrigel (30,000 cells-60,000 cells in 50 μl, and more preferably 50,000 cells in 50 μl) and after solidification, an organoid medium is used to culture organoid. The ratio of sorted cells to matrigel is critical for achieving optimal organoids density for downstream toxin/drug effects evaluation.

Preferably, the culture medium comprises Penicillin/Streptomycin, Amphotericin B, N2 and B27, HEPES, N-Acetylcysteine, gastrin, and the following growth factors: mEGF, FGF10, HGF, Nicotinamide, A83.01, and FSK, Noggin, R-Spondin 1, Wnt3a, Y27632. Frequency of medium is changed every 2, 3, 4 or 5 days, preferably every 3 days. It was found that the medium can be changed every 3 days or more to cut down the medium cost without compromising the organoids growth.

Optionally, the cell culture may comprise additional ingredients such asbile acids (Chenoxycholic acid; Glycochenodeoxycholic Acid; Glycocholic Acid; Taurochenodeoxycholic Acid; Taurocholic Acid; Taurodeoxycholic Acid), extracellular matrix (ECM) proteins of non-BA or BA liver, cytomegalovirus (CMV), immune cells (macrophages; T-cells; B-cells) alone or in combinations.

When a sufficient amount of organoids is generated, usually after 3-10 days, preferably after 5 days, biliatresone at a concentration of 1 μg/ml-10 μg/ml, preferably 2 μg/ml-5 μg/ml was added to organoid cultures, to allow the study of the growth rate, morphology and histology of organoids cultured with or without biliatresone. The effects of biliatresone on the differentiation and functions of human cholangiocytes are discussed below with reference to the examples.

In a preferred embodiment, the organoids are contacted with 2 μg/ml of biliatresone dissolved in a nonaqueous such as DMSO As shown in FIG. 2, at Day 5 post-treatment, the biliatresone-treated organoids are generally small in size (5-20 μm in diameter) and poorly expanded (arrows). In the control well without biliatresone, organoids are larger (25-100 μm in diameter) and well-expanded with a single vacuole.

Some of these organoids display a thick cell layer with multiple vacuoles, displaying a retarded and abnormal growth morphology. In contrast, the control organoids are well expanded and much bigger than biliatresone-treated organoids.

It has been reported that transcriptomic analysis of BA organoids revealed a shift from cholangiocyte to hepatocyte transcriptional signatures (18), and biliatresone has been shown to induce BA-like phenotypes in mouse (15) and zebrafish (12). Results of immunostaining in the present disclosure demonstrate that expression of cholangiocyte marker CK19 is drastically reduced, while hepatocyte marker HFN4A is markedly elevated in biliatresone-treated organoids, indicating an abnormal differentiation towards a hepatocytic lineage.

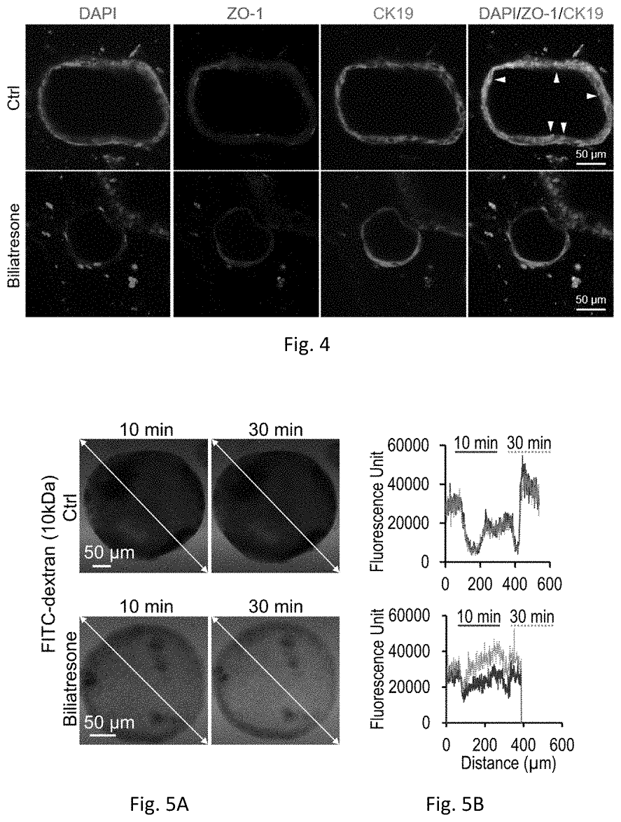

It was previously reported that disturbed apical-basal organization was observed in BA liver organoids (18) as well as in biliatresone-treated mouse cholangiocytes (14). Zona occludens-1 (ZO-1) is a tight junction protein important for the stabilization of the tight junction solute barrier through coupling to the perijunctional cytoskeleton. As shown in FIG. 4, the immunoreactivity of ZO-1 (tight junction marker) is found to be markedly reduced in biliatresone-treated organoids as compared to control.

As shown in FIG. 5, the percentage of Aβ positive cells is significantly higher in biliatresone-treated culture as compared to the control culture. The observed Aβ accumulation in human organoids is consistent with pathobiological and diagnostic feature of BA, whereby altered beta-amyloid-related gene expression, Aβ deposition in liver organoids and in liver tissues; and periductal Aβ deposition are described (18).

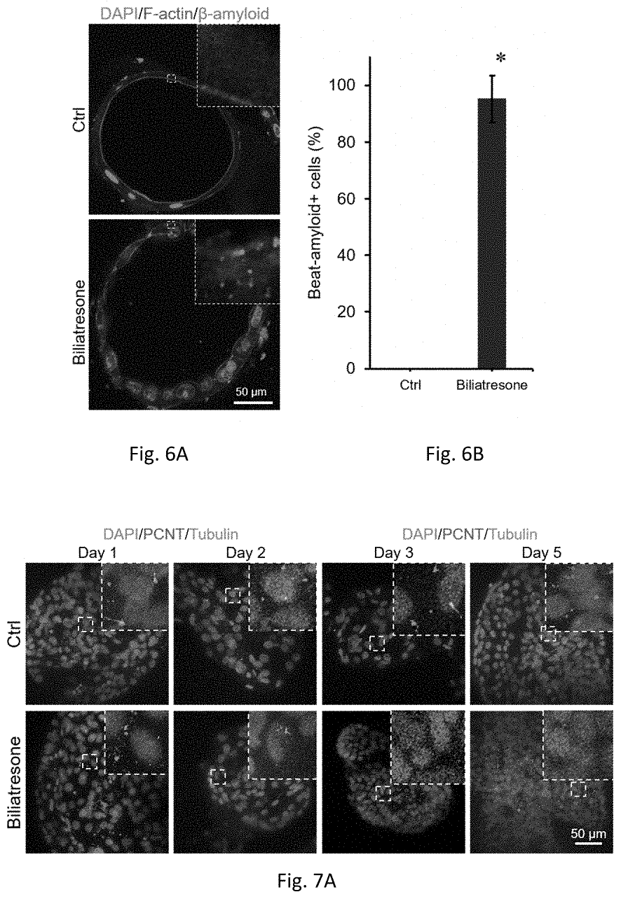

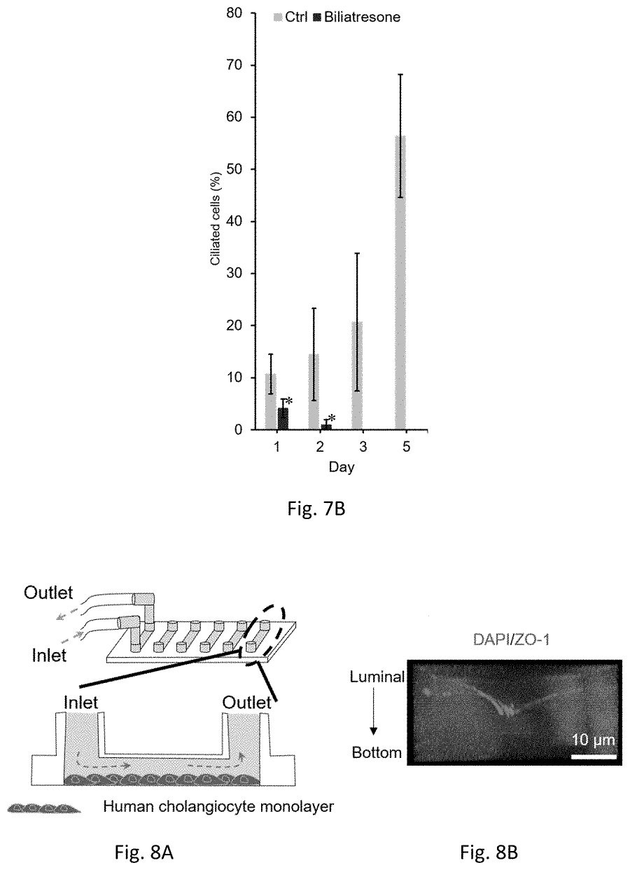

A disrupted F-actin distribution in biliatresone-treated organoids is also shown in FIG. 6. In particular, cytoskeleton protein F-actin is found to ectopically express at both the apical side and basal side in biliatresone-treated organoids. In contrast, F-actin localized at the apical surface of the control organoids. The disrupted F-actin distribution is in line with the perturbed apical-basal polarity in biliatresone-treated organoids as shown by ZO-1 (FIG. 4). Shorter, misoriented, or less abundant cholangiocyte cilia were commonly observed in several studies of both syndromic and non-syndromic BA patients (21-23), suggesting that cilia defects could contribute to BA. In FIG. 7, biliatresone-treated organoids were immuno-stained for ciliary proteins Pericentrin (PCNT) and alpha-tubulin to determine if ciliogenesis in human organoids is disturbed by biliatresone.

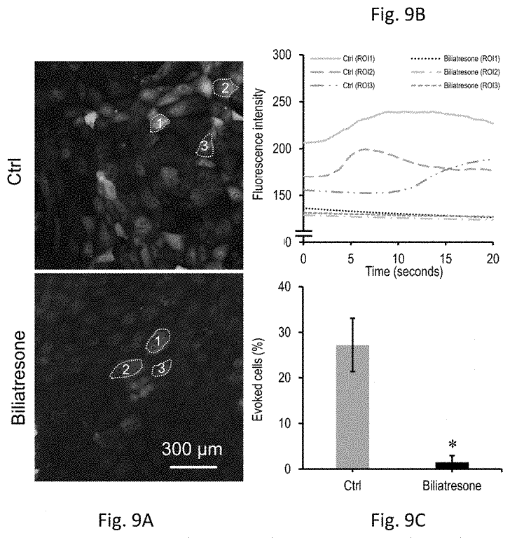

Cholangiocytes of control organoids possess primary cilia and elicited cilia mechano-sensory function. However, the number of ciliated cholangiocytes was significantly reduced (FIG. 7B) and cilia mechano-sensory function was severely hampered in the biliatresone-treated liver organoids (FIG. 9).

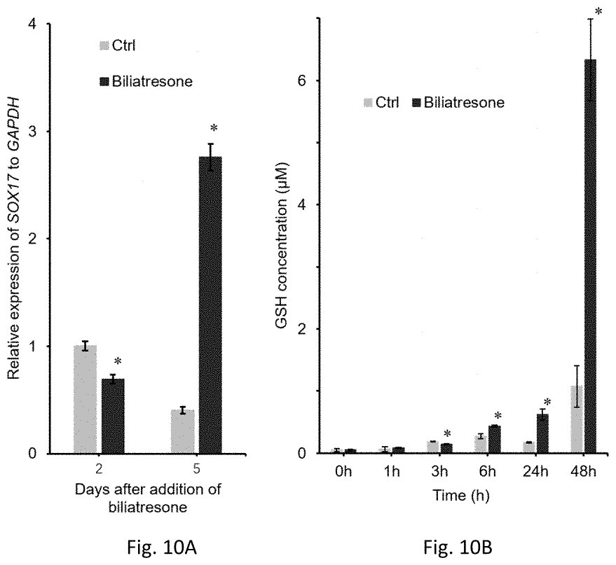

It has been reported that biliatresone decreased the cellular antioxidant glutathione (GSH) and SOX17 level of mouse cholangiocytes (14). GSH and SOX17 are essential for the stabilization of cellular cytoskeleton, which plays a vital role in maintaining the cellular polarity and in ciliogenesis (12, 14). SOX17 is expressed in the endoderm from the onset of gastrulation and plays essential roles in cholangiocyte and bile duct development (31, 32). Sox17 heterozygous mice develop BA-like phenotype as the gallbladder epithelium becomes detached from the luminal wall, which indicates that Sox17 is required to maintain the epithelial architecture of the gallbladder and cystic duct (31). A small but significant drop of SOX17 expression in day-2 biliatresone-treated organoids was detected (FIG. 10), which was associated with a perturbation of cytoskeleton, apical basal polarity and ciliogenesis defects, and loss of epithelial integrity in human cholangiocytes as revealed by abnormal F-actin, α-tubulin and ZO-1 immunofluorescence (FIG. 5-7).

A small but significant drop of GSH level in biliatresone-treated organoids at 3 hours was observed, but the GSH level was restored in biliatresone-treated organoids at 6, 24 and 48 hours (FIG. 10). Biliatresone treatment and/or decreases in GSH level upregulated RhoU/Wrch1, which then mediated an increase in Hey2 in the NOTCH signaling pathway, causing downregulation of the transcription factor Sox17 (33). GSH levels were shown to drop in the first few hours, but were increased and recovered to normal level at 24 hours of biliatresone treatment. However, Sox17 expression was still significantly lower in biliatresone-treated mouse cholangiocytes at 24 hours of treatment (14). Taken all these together suggested that a transient drop of GSH level is sufficient to induce a downregulation of SOX17, and a later recovery of GSH level is unable to restore the normal SOX17 expression.

All of the experimental results confirm that the organoids and bile duct organoids disclosed in the present disclosure resemble biliary atresia-specific morphology.

Generation of Mini-Bile Duct

Organoid cells were seeded on Matrigel-coated microfluidic chip (5,000 of organoid cells per each channel of the Matrigel-coated 6-channel ibidi microfluidic chips (80607, ibidi)), incubated at 37° C. for 2-10 days, preferably 2-5 days, and more preferably 3 days to form the monolayer of mini-bile duct.

Applications of In Vitro Models

The present disclosure demonstrates that addition of biliatresone is able to induce in human liver organoids (in a non-diseased state) morphological and developmental changes found in BA organoids. The biliatresone-induced morphological and developmental changes may provide important insights into the BA pathogenesis in humans, and may lead to the development of new preventive measures for environmental toxin or new treatments.

This in vitro human BA model can be used for toxin screening and pre-clinical testing of new drugs to improve treatment outcome. As compared to cell lines and animal models, human derived cells offer a better solution for mimicking native physiology, modeling diseases, and performing drug screening.

In vitro models can be used as a platform for toxin screening and anti-toxin therapeutic development in accordance with methods and assays known to those skilled in the art. For example, BA-specific cells may be used as a control in experiments where both treated and untreated cells are exposed to a candidate toxin. Alternatively, the in vitro cells may be exposed to a known toxin in combination with potential anti-toxin compounds to study the anti-toxicity of a therapeutic. Those skilled in the art would appreciate that a number of parameters can be selected for measurement in order to quantify the effect of toxins and the efficacy of a candidate therapeutic. Examples of such parameters are growth rate, apoptosis, organoid polarity, organoid transportation, marker expression, functional maturity, and the like.

The disclosed platform systems can offer unique advantages, allowing direct, specific and instant effects of the toxin and anti-toxin therapeutics on the physiological functions of bile duct to be reproducibly assayed and quantified in a real-time fashion.

Other unique advantages include: (1) The effects of toxins on the physiological barrier properties of bile duct can be investigated by transport assay on the organoids platform; (2) Mini bile duct platform allows us to quantify the effect of bile duct toxins such as biliatresone on the calcium signaling of human cholagiocytes; and (3) Real-time fluorescence microscopic imaging of the platform allows continuous, non-invasive and real-time monitoring/quantification of bile duct's responses to toxins and anti-toxin therapeutics.

Therefore, the disclosed models are able to improve the efficacies and throughput of toxicity screening and anti-toxicity treatment development.

### EXAMPLES

### Example 1: Generation of Biliatresone Treated Human Liver Organoids

Wedge liver biopsies (2-3 mm³) of children with non-BA liver diseases including non-tumor liver of hepatoblastoma and choledochal cyst were obtained for liver organoids generation following previously described protocol (18).

Briefly, liver biopsy was minced and digested in gentle MACS-C Tube with 5 ml digestion medium (Multi Tissue Dissociation Kit 1), filtered (70 μm and 30 μm) and sorted by human CD326 (EpCAM) magnetic beads. CD326 positive cells were mixed with Matrigel (50,000 cells in 50 μl) and added to each well of a four-well culture plate (Nunc™ 4-Well Dishes). After Matrigel solidification, organoid medium (Advanced DMEM/F12 supplemented with 1% Penicillin/Streptomycin (Invitrogen), 250 ng/ml Amphotericin B (GIBCO), 25 μM HEPES, 1% N2 and 1% B27(GIBCO), 1.25 mM N-Acetylcysteine (Sigma), 10 nM gastrin (Sigma), and the growth factors: 50 ng/ml mEGF (Peprotech), 100 ng/ml FGF10 (Peprotech), 25 ng/ml HGF (Peprotech), 10 mM Nicotinamide (Sigma), 5 μM A83.01 (Tocris), and 10 μM FSK (Tocris), 25 ng/ml Noggin (Peprotech), 500 ng/ml R-Spondin 1 (R&D), 100 ng/ml Wnt3a (R&D), 10 μM Y27632 (Sigma Aldrich)) was added for organoid culturing. The medium was changed every three days. After culturing for 5 days, biliatresone solution (2867, Axon MEDCHEM; 2 μg/ml in DMSO) was added into the medium and the organoids were cultured for different periods for subsequent analysis. For the untreated control culture, DMSO (same volume as biliatresone) was added to the culture. FIG. 1 is a schematic diagram of the generation of biliatresone treated human liver organoids.

### Example 2: Biliatresone Induced Aberrant Growth of Human Liver Organoids

Hematoxylin and Eosin staining were carried out for the cultured organoids. FIG. 2 are bright-field images showing Hematoxylin and Eosin staining of sections of the control organoids and day-5 biliatresone-treated organoids.

As shown in FIG. 2, control organoids were expanded and developed into well-expanded spherical shape (25-100 μm in diameter) with a single-cell layer of epithelial cells and a single vacuole inside (Ctrl; with DMSO) from day 0 to day 5.

In contrast, there were fewer organoids in the day 5 culture with biliatresone, the organoids were generally smaller (5-20 μm in diameter), not well-expanded (arrows), or very tiny, poorly expanded, and thick cell layer with multiple vacuoles (arrowheads).

### Example 3: Biliatresone Induced Hepatocytic Differentiation of Human Liver Organoids

Immunostaining was carried out to determine whether biliatresone perturb the differentiation of cholangiocyte and hepatocyte in human liver organoids.

Hepatocytic differentiation in human organoids was assessed by immuno-fluorescence for hepatocyte maker (HNF4A). Organoids in the Matrigel were washed with PBS (phosphate-buffered saline) before being fixed with 4% paraformaldehyde at room temperature for 20 minutes. The organoids were permeabilized with Triton X-100 (0.5% in PBS) for 20 minutes and blocked with 3% bovine serum albumin (BSA, Sigma-Aldrich) in PBS with 0.05% Triton X-100 (PBST) for 30 minutes at room temperature. The organoids were then incubated in primary antibodies (diluted in PBST with 3% BSA) at 4° C. overnight. After PBST washes (5 minutes for 3 times), organoids were incubated in secondary antibodies (diluted in PBST with 3% BSA) for 2 hours at room temperature. Afterward, the organoids were rinsed with PBS 3 times and incubated with DAPI solution for 30 minutes at room temperature. The organoids were rinsed with PBS (5 minutes for 3 times). Primary and secondary antibodies used in this examples were anti-ZO-1 (1:50, RA231621, Thermo Fisher), anti-CK19 (1:200, ab220193, Abcam), anti-F-actin (1:500, A22287, Thermo Fisher), anti-beta-amyloid (1:200, SIG-39220, 4G8, BioLegend), anti-HNF4A (1:50, ab201460, Abcam), Alexa Fluor 488 (1:500, A11008, Invitrogen) and Alexa Fluor 594 (1:500, A11032, Invitrogen).

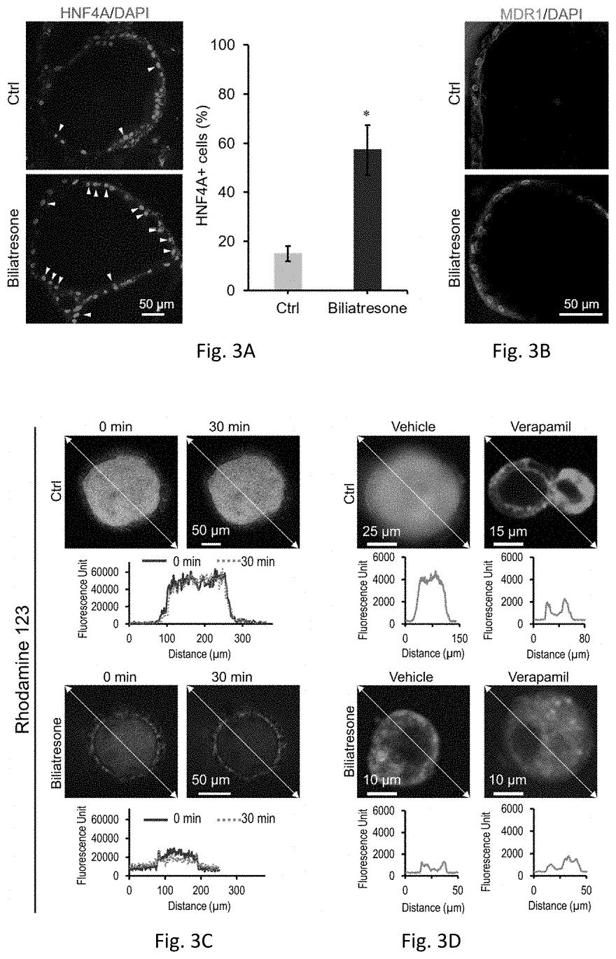

Organoids were imaged on a laser confocal microscopy (Leica) equipped with a 20× dry objective and a 63× oil-immersion objective. Images were analyzed using ImageJ software. For quantification of the number of cilia from each group, the organoids were imaged using confocal microscopy (0.8 μm Z-axis interval, 40 μm in thickness). Maximum intensity projection was performed for visualization of the cilia in the organoids. FIG. 3A are representative images of control and day-2 biliatresone-treated human liver organoids stained for HNF4A (green). Nuclei were stained with DAPI (blue). The percentage of HNF4A positive cells in control and day-2 biliatresone-treated organoids were determined by counting the total number of cells and HNF4A+ cells of the organoids. Number of organoids counted in each group=3; *, p<0.05, student's t-test; error bars indicated the standard deviation.

As shown in FIG. 3A, expression of HFN4A was only detected in very few cells in the control organoids, in contrast, HNF4A expression was markedly elevated in many cells in biliatresone-treated organoids. The results show that the percentage of HNF4A+ cells in organoids increased significantly from 14.9±2% (Mean±SD) in control organoids to 57.3±8% (Mean±SD) in biliatresone-treated organoids.

FIG. 3B are representative images of control and day-2 biliatresone-treated human liver organoids stained for MDR1 (red). Nuclei were stained with DAPI (blue). As shown in FIG. 3B, the expression of cholangiocyte MDR1 was drastically reduced in biliatresone-treated organoids.

A MDR1 activity assay (MDR1-mediated Rhodamine 123 (R123) transport assay) was then performed to investigate the defective cholangiocyte differentiation of biliatresone-treated organoids.

In particular, liver organoids were released from Matrigel by cold Advanced DMEM/F12 and resuspended in culture medium with or without Verapamil (10 μM) and incubated at 37° C. for 30 minutes. Afterward, the liver organoids were washed with PBS and incubated in culture medium containing Rhodamine 123 (100 μM) at 37° C. for 30 minutes. After washing with PBS, the fluorescence of liver organoids was then visualized by laser confocal microscopy (Leica) immediately (as 0 minutes) and after 30 minutes incubation in culture medium.

FIG. 3C includes representative images demonstrating the fluorescent substrate Rhodamine 123 (R123) localization in the lumen of control organoids at 0 and 30 minutes. In contrast, luminal R123 localization of day-2 biliatresone-treated organoids was minimal during the 30 minutes incubation. The fluorescent intensity along the white line of images of control and day-2 biliatresone-treated organoids at 0 and 30 minutes of incubation were plotted.

As shown in FIG. 3C, control organoids were able to transport R123 from the medium into the organoids in an efficient manner. In contrast, biliatresone-treated organoids were very inefficient in transporting R123 across the organoid membrane and only a low level of R123 was accumulated within the organoids after 30 minutes of incubation.

FIG. 3D are representative images demonstrating the inhibition of MDR1 fluorescent substrate R123 localization in the lumen of normal organoids by Verapamil.

In contrast, the minimal luminal R123 localization of day-2 biliatresone-treated organoids was affected with or without the presence of Verapamil. The fluorescent intensity along the white line of images of control and day-2 biliatresone-treated organoids in the presence or absence of Verapamil were plotted.

MDR1 inhibitor Verapamil inhibited the MDR1-mediated R123 transport in control organoids. The low-level accumulation of R123 in biliatresone-treated organoids as shown in FIG. 3D suggested that biliatresone could induce the defective cholangiocyte differentiation of human liver organoids.

### Example 4: Biliatresone Induced Apical-Basal Polarity Defect

To test if biliatresone induces apical-basal polarity defects in human organoids, the control group and biliatresone-treated organoids were stained with anti-ZO-1 antibody. FIG. 4 depicts the result of immunostaining of control and day-2 biliatresone-treated human liver organoids for ZO-1 (green) and CK19 (red). Nuclei were stained with DAPI (blue).

As shown, in the control organoids, ZO-1 formed a smooth layer at the apical side of the cells as expected (FIG. 4; arrowheads), but ZO-1 immuno-reactivity was markedly reduced and without a clear apical predominance in biliatresone-treated organoids. Zona occludens 1 (ZO-1) stabilizes the tight junction solute barrier through coupling to the perijunctional cytoskeleton, and depletion of ZO-1 leads to defects in the barrier for large solutes (20).

In order to determine the integrity of liver organoids, a FITC-labeled dextran diffusion test was carried out to investigate if biliatresone-treated organoids has defects in the tight junction barrier for solutes. The liver organoids were released from Matrigel by cold Advanced DMEM/F12 and resuspended in culture medium containing FITC-labeled dextran of 10 kDa (10 μM). The fluorescent intensity of liver organoids was imaged using laser confocal microscopy at 10 and 30 minutes.

As shown in the left panel of FIG. 5A, FITC-dextran was found to localize in the lumen of day-2 biliatresone-treated organoids after 10 minutes of incubation and continued to accumulate in the incubation period. In contrast, FITC-dextran was not observed in control organoids at 10 and 30 minutes of incubation. The fluorescent intensity along the white line of images of control and day-2 biliatresone-treated organoids at 10 and 30 minutes of incubation were plotted and shown in the right panel of FIG. 5A.

These characterizations demonstrated the compromised structural barrier caused by the biliatresone in human liver organoids.

### Example 5: Biliatresone Induced Cytoskeleton Defects and a Deposition of β-Amyloid Peptide (an) in Human Liver Organoids

To investigate if biliatresone also induces cytoskeleton defects and Aβ accumulation in human organoids, Immunostaining was carried out for the control and day-2 biliatresone-treated human liver organoids for cytoskeleton protein F-actin (green) and β-amyloid (red). Nuclei were stained with DAPI (blue).

The results are demonstrated in FIG. 6; boxed regions were magnified and shown as insets. The percentages of β-amyloid positive cells in control and day-2 biliatresone-treated organoids were determined by counting total number of cells and β-amyloid+ cells of the organoids. Number of organoids counted in each group=3; *, p<0.05, student's t-test; error bars indicated the standard deviation.

As shown in FIG. 6, it was found that the percentage of Aβ positive cells was significantly higher in biliatresone-treated culture than that in the control culture. Whilst Aβ positive cells were frequently detected in biliatresone-treated organoids, no Aβ positive cells were detected in the control organoids.

In addition, as shown in FIG. 6, F-actin immunoreactivity localized at the apical side of the control organoids, whereas F-actin localized to both the apical and the basal sides of the biliatresone-treated organoids. Disrupted F-actin distribution in biliatresone-treated organoids was in line with the perturbed apical-basal polarity in biliatresone-treated organoids as shown by ZO-1 (FIG. 4).

### Example 6: Biliatresone Decreased the Number of Ciliated Cholangiocytes in Human Organoids

To investigate if biliatresone disturbed ciliogenesis in human organoids, control organoids and biliatresone-treated organoids were immuno-stained for ciliary proteins Pericentrin (PCNT) and alpha-tubulin. FIG. 7A depicts control organoids and day-1, -2-3 and -5 biliatresone-treated human liver organoids that were stained for pericentrin (PCNT; green) and acetylated α-tubulin (Tubulin; red). Nuclei were stained with DAPI (blue). Boxed regions were magnified and shown as insets. The percentages of ciliated cells in control and biliatresone-treated organoids were determined by counting total number of cells and ciliated cells of the organoids. Number of organoids counted in each group=3; *, p<0.05, student's t-test; error bars indicated the standard deviation.

As shown in FIG. 7B, the percentages of ciliated cells increased gradually in control organoids from 10.7±5% (Mean±SD) at day 1 to 56.4±10% (Mean±SD) at day 5. However, in biliatresone-treated organoids, the percentages of ciliated cells were only 4.2±2% (Mean±SD) at day 1 and dropped to 0% at day 3, which was significantly lower than that of control organoids (FIG. 7B).

### Example 7: Generation of Mini-Bile Duct

Before seeding the organoid cells, 50 μL of 5% of Matrigel in organoid expansion medium was injected into the channels of microfluidic chips. After incubation at 37° C. for 30 minutes, the channels were rinsed with 500 μL of PBS.

The organoids were released from the Matrigel by cold Advanced DMEM. The organoid pellets were obtained by centrifugation at 300 g, 3 minutes. Afterwards, the organoids were digested for 5 minutes at 37° C. via 1 mL of TrypLE Express Enzyme (1×). About 5,000 of organoid cells were seeded into each channel of the Matrigel coated 6-channel ibidi microfluidic chips (80607, ibidi), and the microfluidic chips were incubated at 37° C. in incubator. At day 3, monoloayer of cholangiocytes was formed representing the mini-bile duct with the luminal side facing the medium and the basal side facing the bottom of the microfluidic chips. To prepare for the calcium influx measurement, cholangiocyte monolayer was first incubated with 5 μM of Calbryte 520-AM (ATT) (prepared in Hanks' balanced salt solution, HBSS) for 1 hour at 37° C. Then, the cholangiocytes were washed with HBSS (5 minutes for 3 times). The 6-channel chip was connected to a syringe, which was mounted on a syringe pump to apply shear flow with HBSS at 1 dyne/cm². Time-lapse images were acquired for green fluorescence for 20 seconds (1-second intervals and 10 milliseconds exposure time) using Nikon Ti2e microscope (Nikon, Japan).

### Example 8: Reduction of Cholangiocyte Cilia Mechano-Sensory Response in Biliatresone Treated Mini-Bile Duct

Cilia acts as a mechanical sensor for fluid flow in bile duct, the shear stress could induce an influx of calcium in cholangiocytes. To functionally assay cilia sensory function in cholangiocyte, calcium signal evaluation of human cholangiocytes were performed.

The organoid cells were seeded onto Matrigel-coated 6-channel ibidi microfluidic chips (80607, ibidi) and incubated for 3 days to form a monolayer of cholangiocytes. To test the effect of biliatresone on the mini-bile duct, biliatresone (final concentration: 2 μg/ml; 2867, Axon MEDCHEM; stock solution: 2 mg/ml in DMSO) was added into the medium and the mini-bile ducts were cultured for 5 days with medium changing daily. For the untreated control culture, DMSO (same volume as biliatresone) was added to the culture.

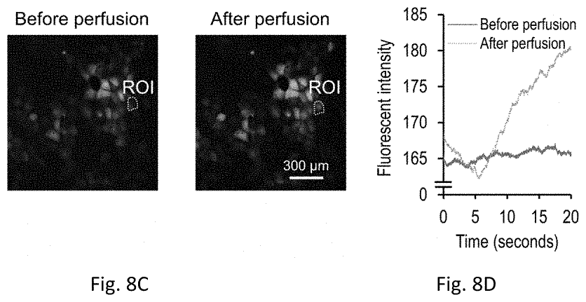

Left panel of FIG. 8A demonstrates the perfusion setup for the microfluidic chip with human cholangiocyte monolayer, and representative image of the cholangiocyte monolayer cultured in the channel of microfluidic chip immunostained for ZO-1 (green) (FIG. 8B). Nuclei were stained with DAPI (blue). ZO-1 staining confirmed that the formation of a cholangiocyte monolayer with their apical sides facing towards the medium.

On day 3 post confluence, cholangiocytes were incubated with 5 μM of Calbryte 520-AM (ATT) (prepared in Hanks' balanced salt solution, HBSS) for 1 hour at 37° C. Before the flow experiment, the cholangiocytes were washed with HBSS (5 minutes for 3 times). The 6-channel chip was connected to a syringe, which was mounted on a syringe pump to apply shear flow with HBSS at 1 dyne/cm2. Time-lapse images were acquired for green fluorescence for 20 seconds (1-second intervals and 10 milliseconds exposure time) using Nikon Ti2e microscope (Nikon, Japan).

FIG. 8C are representative images of cholangiocytes stained with Calbryte 520 before and after perfusion stimulation. FIG. 8D is a plot profile of fluorescent intensity changes of highlighted region (ROI: region of interest) for 20 seconds after perfusion stimulation. As shown, cholangiocytes stayed quiescent under static condition and showed increased calcium influx in response to the medium flow.

The cholangiocyte monolayer was used to address if cilia mechano-sensory function was disrupted after biliatresone treatment. FIG. 9A depicts control and biliatresone-treated human cholangiocytes stained with Calbryte 520.

FIG. 9A are representative images of biliatresone trarted cholangiocytes and un-treated cholangiocytes (Ctrl) stained with Calbryte 520 after perfusion stimulation. FIG. 9B is a plot profile of fluorescent intensity changes of selected control and biliatresone-treated human cholangiocytes (highlighted with dotted line) for 20 seconds. FIG. 9C is the percentage of evoked cells after perfusion stimulation of control (Ctrl) and biliatresone-treated (BTS) human cholangiocytes were determined by counting total number of cells and stimulated cells (green) at three random chosen fields of the cultures. *, p<0.05, student's t-test; error bars indicated the standard deviation.

As shown in FIG. 9, both the percentage of evoked cells and the extent of calcium influx were significantly reduced in biliatresone-treated cholangiocytes than those in untreated cholangiocytes.

### Example 9: Biliatresone Causes Reduction of SOX17 Expression

Gene expressions in liver ductal organoids with or without biliatresone treatment for 2 day and 5 days were investigated. mRNA expression levels were characterized by real-time PCR for SOX17 normalized to GAPDH. The results are shown in FIG. 10A.

GSH levels were measured in liver organoids from 0 to 48 hours without (Ctrl) or with biliatresone. Data was obtained from 3 wells in each group; *, p<0.05, student's t-test; error bars indicated the standard deviation. The results are shown in FIG. 10B.

The above embodiments are described by way of example only. Many variations are possible without departing from the scope of the disclosure as defined in the appended claims.

Although a variety of examples and other information was used to explain aspects within the scope of the appended claims, no limitation of the claims should be implied based on particular features or arrangements in such examples, as one of ordinary skill would be able to use these examples to derive a wide variety of implementations. Further and although some subject matter may have been described in language specific to examples of structural features and/or method steps, it is to be understood that the subject matter defined in the appended claims is not necessarily limited to these described features or acts. For example, such functionality can be distributed differently or performed in components other than those identified herein. Rather, the described features and steps are disclosed as examples of components of systems and methods within the scope of the appended claims.

### REFERENCES

1. Sanchez-Valle A, Kassira N, Varela V C, Radu S C, Paidas C, Kirby R S. Biliary Atresia: Epidemiology, Genetics, Clinical Update, and Public Health Perspective. Adv Pediatr 2017; 64:285-305.
2. Hartley J L, Davenport M, Kelly D A. Biliary atresia. Lancet 2009; 374:1704-1713.
3. Tam P K H, Yiu R S, Lendahl U, Andersson E R. Cholangiopathies—Towards a molecular understanding. EBioMedicine 2018; 35:381-393.
4. Jain V, Burford C, Alexander E C, Sutton H, Dhawan A, Joshi D, Davenport M, et al. Prognostic markers at adolescence in patients requiring liver transplantation for biliary atresia in adulthood. J Hepatol 2019; 71:71-77.
5. Girard M, Panasyuk G. Genetics in biliary atresia. Curr Opin Gastroenterol 2019; 35:73-81.
6. Garcia-Barcelo M M, Yeung M Y, Miao X P, Tang C S, Cheng G, So M T, Ngan E S, et al. Genome-wide association study identifies a susceptibility locus for biliary atresia on 10q24.2. Hum Mol Genet 2010; 19:2917-2925.
7. Leyva-Vega M, Gerfen J, Thiel B D, Jurkiewicz D, Rand E B, Pawlowska J, Kaminska D, et al. Genomic alterations in biliary atresia suggest region of potential disease susceptibility in 2q37.3. Am J Med Genet A 2010; 152A:886-895.
8. Cheng G, Tang C S, Wong E H, Cheng W W, So M T, Miao X, Zhang R, et al. Common genetic variants regulating ADD3 gene expression alter biliary atresia risk. J Hepatol 2013; 59:1285-1291.
9. Chen Y, Gilbert M A, Grochowski C M, McEldrew D, Llewellyn J, Waisbourd Zinman O, Hakonarson H, et al. A genome-wide association study identifies a susceptibility locus for biliary atresia on 2p16.1 within the gene EFEMP1. PLoS Genet 2018; 14:e1007532.
10. Berauer J P, Mezina A I, Okou D T, Sabo A, Muzny D M, Gibbs R A, Hegde M R, et al. Identification of Polycystic Kidney Disease 1 Like 1 Gene Variants in Children With Biliary Atresia Splenic Malformation Syndrome. Hepatology 2019.
11. Brindley S M, Lanham A M, Karrer F M, Tucker R M, Fontenot A P, Mack C L. Cytomegalovirus-specific T-cell reactivity in biliary atresia at the time of diagnosis is associated with deficits in regulatory T cells. Hepatology 2012; 55:1130-1138.
12. Lorent K, Gong W, Koo K A, Waisbourd-Zinman O, Karjoo S, Zhao X, Sealy I, et al. Identification of a plant isoflavonoid that causes biliary atresia. Sci Tranl Med 2015; 7:286ra267.
13. Wang J, Xu Y, Chen Z, Liang J, Lin Z, Liang H, Xu Y, et al. Liver Immune Profiling Reveals Pathogenesis and Therapeutics for Biliary Atresia. Cell 2020; 183:1867-1883 e1826.
14. Waisbourd-Zinman O, Koh H, Tsai S, Lavrut P M, Dang C, Zhao X, Pack M, et al. The toxin biliatresone causes mouse extrahepatic cholangiocyte damage and fibrosis through decreased glutathione and SOX17. Hepatology 2016; 64:880-893.
15. Yang Y, Wang J, Zhan Y, Chen G, Shen Z, Zheng S, Dong R. The synthetic toxin biliatresone causes biliary atresia in mice. Lab Invest 2020.
16. Huch M, Dorrell C, Boj S F, van Es J H, Li V S, van de Wetering M, Sato T, et al. In vitro expansion of single Lgr5+ liver stem cells induced by Wnt-driven regeneration. Nature 2013; 494:247-250.
17. Hu H, Gehart H, Artegiani B, C LO-I, Dekkers F, Basak O, van Es J, et al. Long-Term Expansion of Functional Mouse and Human Hepatocytes as 3D Organoids. Cell 2018; 175:1591-1606 e1519.
18. Babu R O, Lui V C H, Chen Y, Yiu R S W, Ye Y, Niu B, Wu Z, et al. Beta-amyloid deposition around hepatic bile ducts is a novel pathobiological and diagnostic feature of biliary atresia. J Hepatol 2020; 73:1391-1403.
19. Andersson E R, Chivukula I V, Hankeova S, Sjoqvist M, Tsoi Y L, Ramskold D, Masek J, et al. Mouse Model of Alagille Syndrome and Mechanisms of Jagged1 Missense Mutations. Gastroenterology 2018; 154:1080-1095.
20. Van Itallie C M, Fanning A S, Bridges A, Anderson J M. ZO-1 stabilizes the tight junction solute barrier through coupling to the perijunctional cytoskeleton. Mol Biol Cell 2009; 20:3930-3940.
21. Karjoo S, Hand N J, Loarca L, Russo P A, Friedman J R, Wells R G. Extrahepatic cholangiocyte cilia are abnormal in biliary atresia. J Pediatr Gastroenterol Nutr 2013; 57:96-101.
22. Frassetto R, Parolini F, Marceddu S, Satta G, Papacciuoli V, Pinna M A, Mela A, et al. Intrahepatic bile duct primary cilia in biliary atresia. Hepatol Res 2018; 48:664-674.
23. Chu A S, Russo P A, Wells R G. Cholangiocyte cilia are abnormal in syndromic and non-syndromic biliary atresia. Mod Pathol 2012; 25:751-757.
24. Masyuk A I, Masyuk T V, Splinter P L, Huang B Q, Stroope A J, LaRusso N F. Cholangiocyte cilia detect changes in luminal fluid flow and transmit them into intracellular Ca2+ and cAMP signaling. Gastroenterology 2006; 131:911-920.
25. Larusso N F, Masyuk T V. The role of cilia in the regulation of bile flow. Dig Dis 2011; 29:6-12.
26. Gradilone S A, Masyuk A I, Splinter P L, Banales J M, Huang B Q, Tietz P S, Masyuk T V, et al. Cholangiocyte cilia express TRPV4 and detect changes in luminal tonicity inducing bicarbonate secretion. Proc Natl Acad Sci USA 2007; 104:19138-19143.
27. Waters A M, Beales P L. Ciliopathies: an expanding disease spectrum. Pediatr Nephrol 2011; 26:1039-1056.
28. Masyuk A I, Huang B Q, Radtke B N, Gajdos G B, Splinter P L, Masyuk T V, Gradilone S A, et al. Ciliary subcellular localization of TGRS determines the cholangiocyte functional response to bile acid signaling. Am J Physiol Gastrointest Liver Physiol 2013; 304:G1013-1024.
29. Hall C, Sato K, Wu N, Zhou T, Kyritsi K, Meng F, Glaser S, et al. Regulators of Cholangiocyte Proliferation. Gene Expr 2017; 17:155-171.
30. Hartley J L, O'Callaghan C, Rossetti S, Consugar M, Ward C J, Kelly D A, Harris P C. Investigation of primary cilia in the pathogenesis of biliary atresia. J Pediatr Gastroenterol Nutr 2011; 52:485-488.
31. Uemura M, Hara K, Shitara H, Ishii R, Tsunekawa N, Miura Y, Kurohmaru M, et al. Expression and function of mouse Sox17 gene in the specification of gallbladder/bile-duct progenitors during early foregut morphogenesis. Biochem Biophys Res Commun 2010; 391:357-363.
32. Kanai-Azuma M, Kanai Y, Gad J M, Tajima Y, Taya C, Kurohmaru M, Sanai Y, et al. Depletion of definitive gut endoderm in Sox17-null mutant mice. Development 2002; 129:2367-2379.
33. Fried S, Gilboa D, Har-Zahav A, Lavrut P M, Du Y, Karjoo S, Russo P, et al. Extrahepatic cholangiocyte obstruction is mediated by decreased glutathione, Wnt and Notch signaling pathways in a toxic model of biliary atresia. Sci Rep 2020; 10:7599.

1. An in vitro liver organoid model of biliary artresia comprising biliatresone-treated liver organoids and/or mini-bile ducts.

2. The in vitro liver organoid model according to claim 1, wherein the liver organoids and/or mini-bile ducts are characterized by one or more characteristics selected from the group consisting of retarded growth; disturbed apical-basal organization; defective cholangiocyte development; β-amyloid (Aβ) accumulation; reduction of primary cilia and cilia mechano-sensory function of cholangiocytes.

3. The in vitro liver organoid model according to claim 1, wherein the biliatresone-treated liver organoids and/or mini-bile ducts are characterized by one or more characteristics relative to the untreated cells, wherein the characteristics are selected from the group consisting of:
reduced expression of cholangiocyte marker CK19;
increased expression of marker HFN4A;
reduced ZO-1 immunoreactivity;
ectopic expression of F-actin at both of the apical and basal sides;
reduced primary cilia; and
reduced cilia mechano-sensory function of cholangiocytes.

4. The in vitro liver organoid model according to claim 1, wherein the biliatresone-treated liver organoids and/or mini-bile ducts are derived from a non-diseased liver tissue or human induced pluripotent stem cells (IPSCs).

5. The in vitro liver organoid model according to claim 1, wherein the liver organoids and/or mini-bile ducts are obtained by the following steps:
a. obtaining liver tissues from non-diseased human subjects;
b. culturing CD326 positive cells in an extracellular protein matrix in the presence of a culture medium;
c. contacting the cells obtained from the culturing step with biliatresone,
or wherein the liver organoids and/or mini-bile ducts are obtained by the following steps.
a. generating human induced pluripotent stem cells (hIPSCs) from peripheral blood;
b. differentiating hIPSCs to liver organoids;
c. contacting the liver organoids obtained from the differentiating step with biliatresone.

6. The in vitro liver organoid model according to claim 5, wherein the liver organoids and/or mini-bile ducts are contacted with biliatresone at a concentration of 1 μg/ml-10 μg/ml, preferably 2 μg/ml-5 μg/ml.

7. The in vitro liver organoid model according to claim 5, wherein the liver organoids are contacted with bilitresone for 1 to 10 days, preferably 3 to 5 days.

8. The in vitro liver organoid model according to claim 1, wherein the model is a three-dimensional (3D) liver organoid model of biliary artresia.

9. The in vitro liver organoid model according to claim 1, wherein the model is a three-dimensional (3D) liver organoid model comprising only one or more organoid(s) derived from liver or hIPSCs.

10. The in vitro liver organoid model according to claim 1, wherein the model comprises both liver organoids and mini-bile ducts derived from liver or hIPSCs.

11. The in vitro liver organoid model according to claim 1, wherein the model comprises additional cell types selected from liver cells, vascular cells and immune cells.

12. A method of generating in vitro liver organoid model of biliary artresia comprising biliatresone-treated liver organoids and/or mini-bile ducts, the method comprises the steps of:
a. obtaining human liver tissue from subjects free of liver diseases;
b. digesting the tissue and selecting CD326 positive cells;
c. culturing CD326 positive cells in an extracellular protein matrix in the presence of a culture medium; and
d. contacting the cells obtained from the culturing step with biliatresone.

13. The method of claim 12, wherein the liver organoids and/or bile duct organoids are contacted with biliatresone at a concentration of 1 μg/ml-10 μg/ml, preferably 2 μg/ml-5 μg/ml.

14. The method of claim 1, wherein the liver organoids are contacted with bilitresone for 1 to 10 days, preferably 3 to 5 days.

15. The method of claim 1, wherein the culture medium comprising DMEM/F12, HEPES, Penicillin/Streptomycin, Amphotericin B, N2, B27, N-Acetylcysteine, gastrin, and growth factors: mEGF, FGF10, HGF, Nicotinamide, A83.01, FSK, Noggin, R-Spondin 1, Wnt3a, and Y27632.

16. Use of the in vitro liver organoid model of biliary artresia according to claim 1 as a platform for screening for a toxin.

17. Use of the in vitro liver organoid model of biliary artresia according to claim 1 as a platform for screening for an anti-toxin therapeutic.

18. A method of determining the effect of a test compound comprising the following steps:
a. providing the in vitro liver organoid model of biliary artresia according to claim 1;
b. contacting the in vitro liver organoid model of biliary artresia with the test compound.
c. determining the effect of the test compound on the liver organoids and/or min-bile ducts.

19. The method according to claim 18, wherein the step of determining the effect of the test compound comprises quantification of one or more of the following parameters: growth rate, apoptosis, organoid polarity, organoid transportation, marker expression and functional maturity of the cells.
